(19) United States
(12) Patent Application Publication
(10) Pub. No.: US 2013/0079659 A1
(43) Pub. Date: Mar. 28, 2013

(54) INTEGRATION OF ELECTROENCEPHALOGRAPHY (EEG) AND TRANSCRANIAL DIRECT CURRENT STIMULATION (tDCS) WITH HIGH-SPEED OPERATION, ELECTRODE, RE-USE, AUTOMATED tDCS ELECTRODE CONFIGURATION, AND MULTIPLE INDEPENDENT tDCS CURENT SOURCES

(71) Applicants: Elshan Akhadov, Los Alamos, NM (US); Ann Speed, Albuquerque, NM (US); David P. Ryan, Albuquerque, NM (US); Laura E. Matzen, Albuquerque, NM (US)

(72) Inventors: Elshan Akhadov, Los Alamos, NM (US); Ann Speed, Albuquerque, NM (US); David P. Ryan, Albuquerque, NM (US); Laura E. Matzen, Albuquerque, NM (US)

(21) Appl. No.: 13/241,531

(22) Filed: Sep. 23, 2011

(51) Int. Cl. A61B 5/0476

(52) U.S. Cl. 600/544

(57) ABSTRACT

Transcranial direct current stimulation (tDCS) and electroencephalography (EEG) are integrated, including re-using electrodes to perform both tDCS and EEG, and automatically alternating EEG collection and tDCS application. EEG and tDCS functionalities are integrated into a single headset. Improvements in tDCS include realizing multiple tDCS current flow configurations without repositioning electrodes, and concurrently applying multiple independent tDCS currents to a subject.

This invention was developed under Contract DE-AC04-94AL85000 between Sandia Corporation and the U.S. Department of Energy. The U.S. Government has certain rights in this invention.

### FIELD

The present work relates generally to transcranial direct current stimulation and electroencephalography and, more particularly, to integration of the two.

### BACKGROUND

Transcranial direct current stimulation (tDCS) involves applying weak electrical currents to the brain to alter the firing rates of neurons. This is conventionally performed by applying current of 1-2.5 mA between two saline soaked pads positioned in contacting relationship to the scalp, so that current flows over a large portion of the scalp. This technology poses some difficulties. For example, (1) the saline solution tends to drain to the bottom of the pads, causing an uneven current distribution; (2) there is little spatial control; and (3) because of the size of the tDCS pads, there is the possibility that they may stimulate adjacent cortical areas in addition to the intended area.

Because current density is more critical than total current flow in tDCS, one alternative, referred to as High Definition tDCS (FID-tDCS), uses much smaller electrode pads. Whereas typical electrode pads in standard tDCS have a surface area of around 25˜50 cm², the electrode pads of HD-tDCS have a surface area around 1 cm² (diameter under 12 mm). This improves spatial control and helps avoid stimulation of unintended areas. The same current densities achieved with standard tDCS can thus be achieved with significantly smaller currents using HD-tDCS.

Electroencephalography (EEG) involves the use of electrodes to record electrical activity on the scalp caused by neurons firing in the brain. EEG recordings are typically collected in a laboratory using professional equipment. Combined use of EEG and tDCS technologies is desirable because, for example, it provides the capability of observing brain activity before and after application of tDCS, thereby providing measurement of the brain's response to tDCS. Conventional approaches to the combination of EEG and to tDCS involve a relatively cumbersome sequence of procedures including placement of the pair of tDCS pads on the scalp, application of tDCS currents, removal of the tDCS pads, placement of EEG electrodes on the scalp, and subsequent observation of EEG activity.

It is desirable in view of the foregoing to provide for improved integration of EEG and tDCS.

### BRIEF DESCRIPTION OF THE DRAWINGS

FIG. 1 illustrates a prior art headset arrangement that uses a plurality of electrodes to collect EEG data.

FIG. 2 diagrammatically illustrates an apparatus that integrates tDCS and EEG functionalities according to example embodiments of the present work.

FIG. 3 diagrammatically illustrates a switching element of the switching arrangement of FIG. 2 according to example embodiments of the present work.

FIGS. 4 and 5 illustrate respective examples of electrode configurations for tDCS according to the present work.

FIGS. 6 and 7 diagrammatically illustrate examples of constant current sources that may be used in the system of FIG. 2 according to the present work.

FIG. 8 illustrates operations for switching a set of electrodes between tDCS and EEG operating modes according to example embodiments of the present work.

FIG. 9 illustrates operations for automatic leakage current compensation according to example embodiments of the present work.

FIG. 10 diagrammatically illustrates an analog-to-digital converter that permits the system of FIG. 2 to perform automatic leakage current compensation according to example embodiments of the present work.

FIG. 11 illustrates an example of a graphical user interface implemented by the host computer of FIG. 2 according to the present work.

FIG. 12 diagrammatically illustrates a headset that integrates tDCS and EEG functionalities according to example embodiments of the present work.

### DETAILED DESCRIPTION

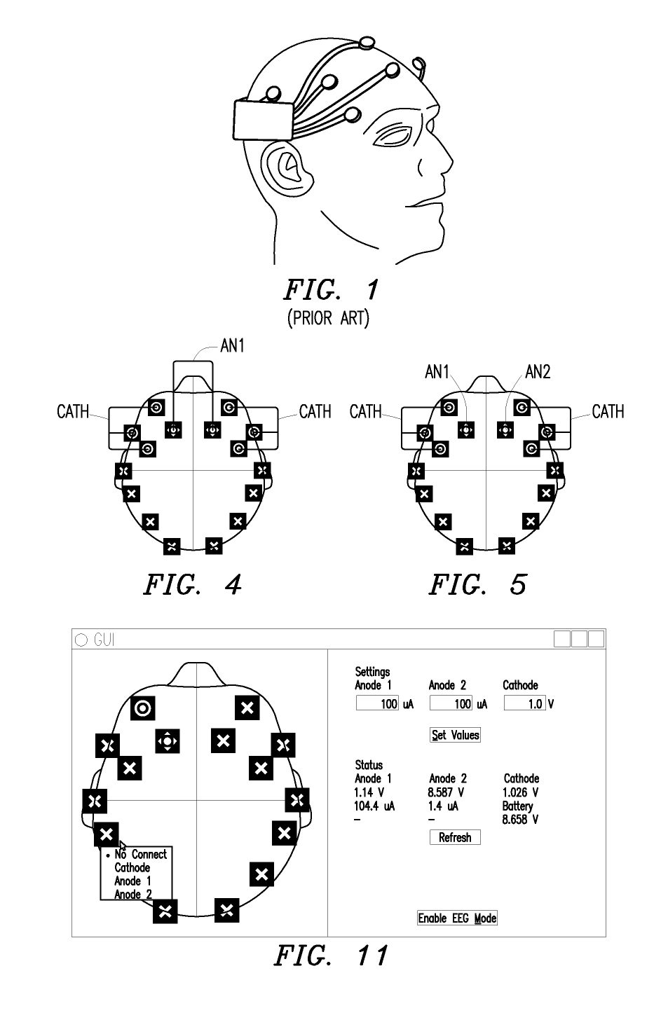

A number of low-cost EEG-like devices have emerged in recent years. One of the most sophisticated of these is the Emotiv EPOC EEG headset, which positions 14 measurement electrodes across the frontal, temporal, and occipital lobes of the brain, and provides four other electrodes for use as references at respective locations. The Emotiv headset, shown in FIG. 1, includes a plastic housing that contains pre-positioned electrodes. The housing is made from a Polycarbonate-ABS blend, which is flexible enough to allow the device to accommodate different sized heads while still maintaining sufficient pressure to keep the electrodes in place. The electrodes, which are to approximately 10.5 mm in diameter, are connected by wires to an analog board provided within the plastic housing, and located above the subject's right ear. The Emotiv headset, typically marketed toward garners as an input device for interacting with a game computer, has been shown to function as an effective EEG device for use in neuropsychological experiments.

Example embodiments of the present work provide a flexible platform integrating tDCS and EEG functionalities. The electrodes provided in various low-cost EEG-like devices (such as the Emotiv headset shown in FIG. 1) are comparable in size to the aforementioned electrode pads used for HD-tDCS. The present work recognizes that this provides an advantageous location to intercept and redirect the electrodes for connection to suitable tDCS circuitry. Some embodiments provide at least two independent precision adjustable constant-current sources, and a plurality of electrodes may be connected to the anodes of the current sources in any tDCS configuration. Some embodiments provide over a billion different electrode configurations for tDCS. The independent current sources permit delivery of matching currents to different electrodes. Some embodiments feature measurement capabilities to verify the tDCS current values and ensure that they are within the voltage compliance of the system.

Some embodiments provide for observation of brain activity immediately before and after tDCS current application, thereby providing measurement of the brain's response to tDCS in a heretofore unknown manner. Some embodiments automatically switch between tDCS mode and EEG mode in less than two microseconds, many orders of magnitude faster than prior art techniques. This remarkable improvement in operating speed is achieved virtually independently of the number of electrodes employed, whereas the speed of the prior art techniques is greatly affected by the number of electrode pads used for tDCS.

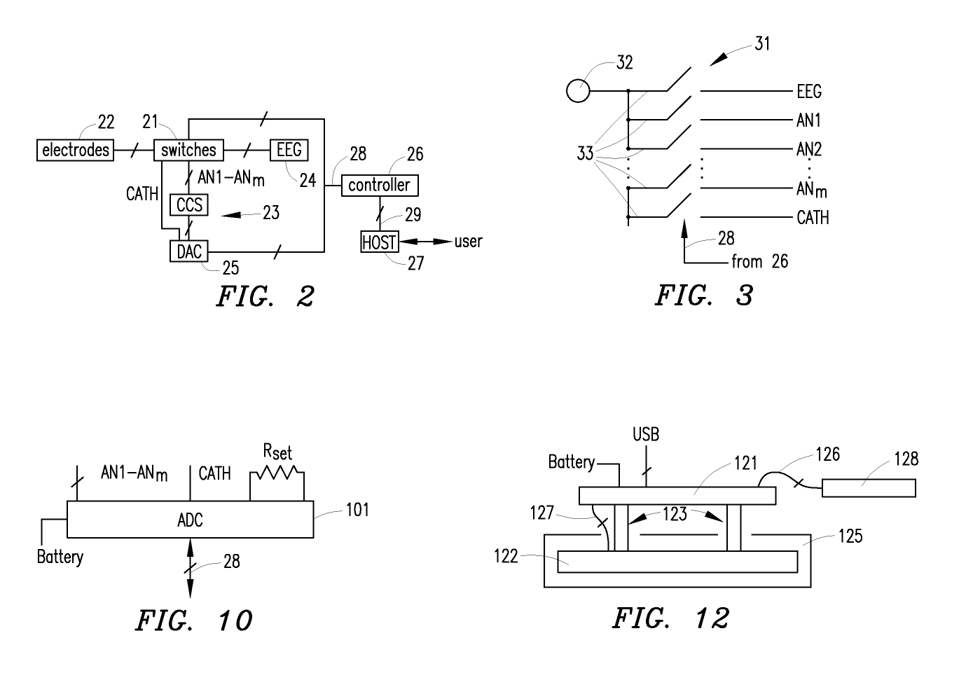

FIG. 2 diagrammatically illustrates an apparatus that integrates tDCS and EEG to functionalities, using the same set of electrodes for both tDCS and EEG, according to example embodiments of the present work. A switching arrangement 21 selectively connects either a tDCS drive arrangement 23 or an EEG analyzer 24 to a set of electrodes 22. A digital controller 26 is coupled to control the switching arrangement 21 via a bus 28. The tDCS drive arrangement 23 includes m independent constant current sources, collectively designated as CCS. A digital-to-analog converter (DAC) 25 is coupled to control the constant current sources, and the digital controller 26 is coupled to control the DAC 25 via the bus 28. A host computer 27 controls the digital controller 26 via a host interface 29. The host computer 27 provides a user interface that receives input from a user and provides output information to the user.

The switching arrangement 21 includes a plurality of switching elements coupled respectively to the electrodes 22 in one-to-one correspondence. FIG. 3 diagrammatically illustrates the structure of the individual switching elements 31 within the switching arrangement 21 according to example embodiments of the present work. As shown in FIG. 3, each switching element 31 within the switching arrangement 21 includes a plurality of single-pole-single-throw (SPST) switches 33 connected to an associated electrode 32 within the set of electrodes 22. The switches 33 selectively connect the electrode 32 to respectively associated nodes, designated as EEG, AN1, AN2, . . . ANm, and CATH. The EEG node is a node of the EEG analyzer 24 normally connected to the associated electrode 32 in an EEG mode of operation. The nodes AN1-ANm (see also FIG. 2) are m current sourcing nodes (also referred to as anodes) respectively provided by the m constant current sources CCS of the tDCS drive arrangement 23. The node CATH is the current sink node (also referred to as the cathode) of the tDCS drive arrangement 23.

Each of the switches 33 of a given switching element 31 may be controlled by the controller 26 independently of the other switches 33 of that switching element, and independently of the other switching elements 31. Thus, the controller 26 may configure to the switching arrangement 21 such that any given electrode 32 is connected by its associated switching element 31 to any of the m+2 nodes shown in FIG. 3, independently of how the other electrodes 32 are connected by their respectively associated switching elements 31. During the EEG mode of operation, all of the electrodes 31 may be connected to their normally associated nodes within the EEG analyzer 24. During a tDCS mode of operation, the switching elements 31 may re-direct connections of the electrodes 32 away from their normally associated EEG nodes, such that any electrode 32 may be connected to any of the tDCS anodes AN1-ANm, or to the tDCS cathode CATH, or may be left unconnected (floating). It is evident that, for example, using an Emotiv headset that has up to 18 electrodes available for tDCS use, the possible electrode configurations for the tDCS mode are manifold.

FIGS. 4 and 5 illustrate two examples of the multitude of possible electrode configurations for tDCS mode according to the present work. In the example of FIG. 4, two electrodes are connected to AN1, six electrodes are connected to CATH, and the remaining electrodes are floating. In the example of FIG. 5, one electrode is connected to AN1, one electrode is connected to AN2, six electrodes are connected to CATH, and the remaining electrodes are floating.

FIGS. 4 and 5 also illustrate an advantage of providing a plurality of constant current sources for tDCS. Referring also to FIG. 2, the anodes AN1-ANm of the m constant current sources CCS are, for example, capable of delivering matching currents to m different electrodes at 22. An advantage of this is demonstrated by comparing FIGS. 4 and 5. In FIG. 4, if AN1 delivers 1 mA, the currents out of the two electrodes connected to AN1 may not be evenly split at 500 μA, each, due to variations in contact resistances among the electrodes and/or variations in scalp resistance. With the configuration of FIG. 5, however, both AN1 and AN2 can be set to deliver 500 μA, which ensures that the currents out of the electrodes connected to AN1 and AN2 are equal. The constant current sources driving AN1 and AN2 will adjust their respective voltages at AN1 and AN2 as necessary so that each anode delivers the desired 500 μA.

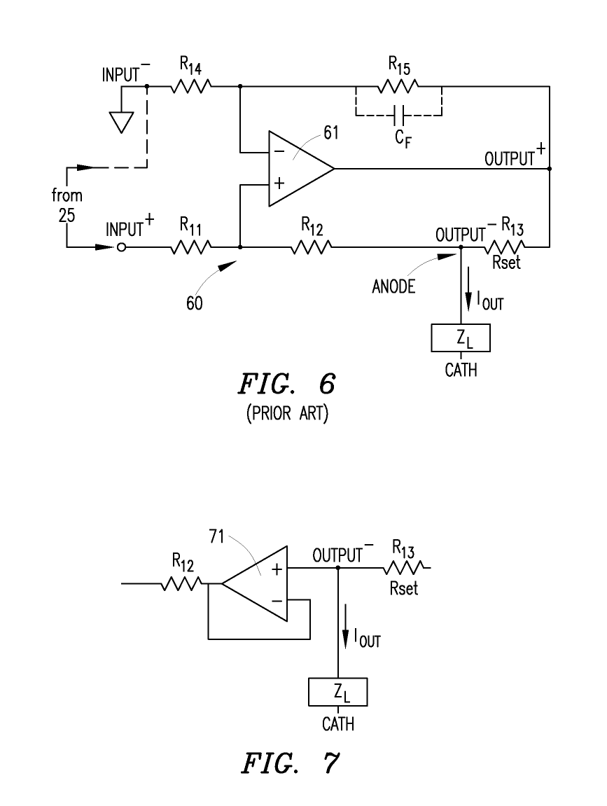

FIG. 6 diagrammatically illustrates in more detail an example of a conventional constant current source 60 suitable for the present work. In some embodiments, m of the FIG. 6 current sources are provided at CCS in FIG. 2. The example constant current source 60 is implemented using the so-called “improved Howland current pump”, which is well-known in the art. The circuit of FIG. 6 may be understood as a unity-gain differential amplifier that “mirrors” the input voltage to the output voltage. That is, Input+−Input−=Output+−Output−. For example, if Rset=1 kΩ, and Input+−Input−=200 mV, then Output+−Output−=200 mV, so the output (anode) current, Iout, is 200 mV/1000Ω=200 μA.

The resistor ratios should be appropriately balanced, such that R11/(R12+R13)=R14/R15. Some embodiments provide, on a printed circuit board where the constant current sources are constructed, several resistor footprints arranged in series and parallel to allow nonstandard trim resistor values to be created using standard SMD resistors. Some embodiments use an LT1991 differential amplifier, conventionally available from Linear Technology Corporation. This amplifier has better than 0.04% matching resistors, and provides sufficient accuracy for keeping the current constant within a few percent without any trimming. When properly tuned, the input voltage to the improved Howland current pump is proportional to the output current, independent of the load impedance, assuming the current source is within its compliance voltage range, and ignoring leakage current through R12 (discussed below). In some embodiments, R11, R12, R14 and R15 are each 450 kΩ, and R13 (Rset)=1 kΩ. As shown by broken line in FIG. 6, some embodiments provide a suitable filter capacitor C_{F} across R15. Some embodiments also provide a similar filter capacitor across R12 (not shown in FIG. 6). The load Z_{L} in FIG. 6 represents the electrode(s) and the scalp as connected in circuit between the anode and the cathode.

Based on the aforementioned relatively low currents required for HD-tDCS, the current source of FIG. 6 may be designed to be precise at low currents (down to 1 μA). The range and resolution of the current source is selectable with the single “set resistor” Rset. For example, in some embodiments, the DAC 25 driving Input+ and Input− is a 16 bit device that outputs from 0 to 2.5V, and Rset is 1 kΩ. This limits Iout to safe levels, e.g., approximately 0-2.5 mA, with resolution of approximately 0.04 μA. If larger currents are required, Rset may be changed to a lower value. For example, in some embodiments, Rset=100Ω, and the resolution is approximately 0.4 μA with a range of approximately of 0-25 mA.

Note that some leakage current flows through resistor R12 instead of into the load. However, if resistors R11, R12, R14 and R15 are much larger than Rset in FIG. 6, the leakage current flowing through R12 is negligible. FIG. 7 illustrates an alternative embodiment that provides a unity gain buffer 71 (with picoampere leakage current) feeding R12 from the Output− node, so that lout is virtually equal to the current through Rset.

Because the output of the differential amplifier 61 (Output+ in FIG. 6) cannot go all the way to ground with a single power supply, some embodiments compensate by using the DAC 25 to maintain the cathode voltage (CATH in FIGS. 2, 6 and 7) slightly above ground (so the anode can be dropped to meet the cathode voltage). An output of the DAC 25 thus functions as the cathode (sinking current into the DAC). In some embodiments, the cathode voltage is set to approximately 100 mV.

Note also that the outputs of the DAC 25 may not go all the way to ground when the DAC is set to zero scale, due to the zero scale offset of the DAC. This prevents the output current lout from going all the way to zero. (Hence the current range would be 1 μA-2.5 mA for lout in the foregoing example with Rset=1 kΩ if the DAC has a zero scale offset of 1 mV.) For applications that may require Iout to go all the way to zero, the input reference voltage, Input−, may be set slightly higher than ground, for example, using an output of DAC 25 to drive Input− to some small positive voltage (e.g., a few mV). This is shown by broken line in FIG. 6.

Referring again to FIG. 2, in some embodiments, the host interface 29 is a conventional USB bus, and the bus 28 is a conventional SPI bus, with the digital controller 26 acting as the SPI master, and the switching arrangement 21 and DAC 25 acting as SPI slaves. In some embodiments, the digital controller 26 is implemented with the FT4232H High-Speed Quad USB UART IC available from Future Technology Devices International Ltd., the switching arrangement 21 is implemented with a suitable number of the ADG1414 (Serially-Controlled Octal SPST Switches) available from Analog Devices, Inc., and the DAC 25 is implemented with a suitable number of the AD5668 Octal 16-bit DAC available from Analog Devices, Inc. The FT4232H has a defined API for executing commands over an SPI bus. In some embodiments, software on the host computer 27 permits a user to configure the electrodes 22 and control the tDCS currents, by using the USB bus 29 to communicate with the controller 26, which in turn controls the switching arrangement 21 and DAC 25 via the SPI bus 28.

In some embodiments, the digital controller 26, constant current sources CCS, DAC 25 and switching arrangement 21 are provided on a first printed circuit board (also referred to a the tDCS board) similar in size to a second printed circuit board (also referred to as the EEG board) that contains the EEG components of the aforementioned Emotiv headset. The EEG board, located above the wearer's ear in FIG. 1, generally corresponds to the EEG analyzer 24 of FIG. 2. Suitable openings are cut into the housing around the EEG board to permit mounting the tDCS board to the EEG board using suitable conventional techniques. Jumper wires are used to insert the switching arrangement 21 electrically between the headset electrodes and the EEG board. This to results in a modified headset that conforms electrically to FIG. 2, and has a physical structure similar to that of the Emotiv headset shown in FIG. 1.

FIG. 12 diagrammatically illustrates the above-described arrangement wherein the tDCS board 121 (containing the digital controller 26, constant current sources CCS, DAC 25 and switching arrangement 21 of FIG. 2) is mounted to the EEG board 122 by suitable mounting structure 123 that extends through openings in the housing 125 that surrounds the EEG board 122. Jumper wires 126 connect the Emotiv electrode cabling 128 to the tDCS board 121, and jumper wires 127 connect the tDCS board 121 to the EEG board 122. In some embodiments, the host computer 27 (see also FIG. 2) is connected to the tDCS board 121 by a USB cable, and the tDCS board 121 is powered by connection to a suitable power supply, such as a 9V or 18V battery.

Various embodiments use various combinations of scalp electrodes and EEG analyzers. For example, in some embodiments, the electrodes 22 of FIG. 2 are provided by a commercially available, disposable headset, and the EEG analyzer 24 is provided by a laboratory-grade EEG apparatus.

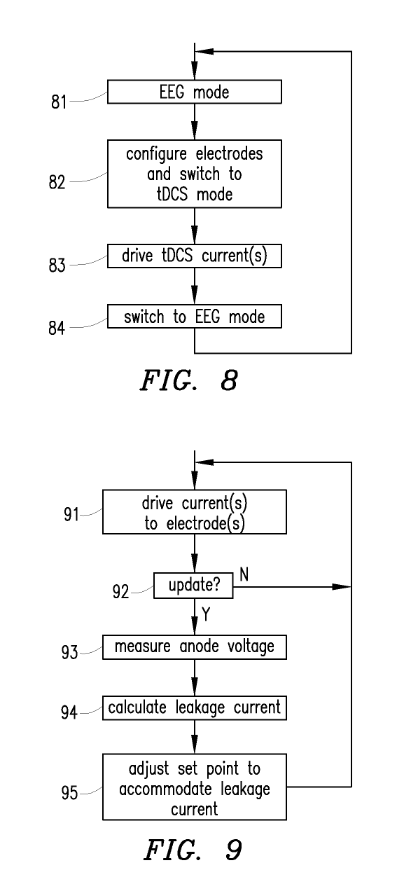

FIG. 8 illustrates operations that may be performed according to example embodiments of the present work. In some embodiments, the operations of FIG. 8 are performed by the system of FIG. 2 automatically, under control of the digital controller 26 and host 27. At 81, with the electrodes connected for EEG mode operation, the electrodes are monitored for EEG purposes as is conventional. At 82, the electrode connections are switched to a desired configuration (see also FIGS. 3 and 4) for tDCS mode operation. At 83, one or more selected tDCS currents are driven to one or more of the electrodes by one or more constant current sources for a desired interval of time. Various embodiments use various tDCS current drive intervals. As one example, the tDCS current drive interval is 15 minutes in some embodiments. When the tDCS current drive operation is completed at 83, the electrode connections are immediately switched to back to EEG mode at 84. As mentioned above, some embodiments switch from tDCS mode to EEG mode in less than two microseconds. After the switch to EEG mode at 84, the electrodes are again monitored for EEG purposes at 81.

In some embodiments, the application of tDCS current includes ramping the output current up to the desired value. In some embodiments, the tDCS current is similarly ramped down to zero. Various embodiments employ various waveforms to effect application and removal of tDCS current.

Some embodiments provide capability to compensate automatically for the aforementioned leakage current that flows through resistor R12 in FIG. 6. To compensate for the leakage current, the anode voltage (at Output−) is measured, and the amount of leakage current flowing to the differential amplifier is calculated as

[(Output−)−(Input+)]/900 k (for R11 and R12 of 450 kΩ).

The set point of the input voltage (Input+−Input−) is then adjusted such that the corresponding output current is equal to the sum of the desired load current and the calculated leakage current. The current flowing into the load then matches the desired current. In some embodiments, the automatic leakage current compensation is updated once per second. Various embodiments update the leakage current compensation at various rates.

FIG. 9 illustrates an example of the aforementioned automatic leakage current compensation according to the present work. The operations of FIG. 9 may be integrated into the tDCS current drive operation 83 of FIG. 8. The operations at 91 and 92 drive the desired output current(s) at 91 until it is determined at 92 that an update interval has expired. When the update interval expires, the anode voltage is measured at 93, after which the leakage current is calculated at 94. At 95, the set point of the input voltage is adjusted such that the corresponding output current is equal to the sum of the desired load current and the calculated leakage current. This adjustment accommodates the effect of the leakage current on the load current.

FIG. 10 diagrammatically illustrates an analog-to-digital converter (ADC) 101 that is cooperable with the digital controller 26 via bus 28 (see also FIG. 2) for implementing automatic leakage current compensation according to example embodiments of the present work. The ADC 101 communicates with the controller 26 on bus 28, and receives the anode voltages AN1-ANm as analog inputs to permit measurement of those voltages. The controller 26 of FIG. 2 periodically reads an anode voltage measurement as provided on bus 28 by ADC 101, calculates the leakage current based on the measurement, and appropriately updates the input voltage set point via the DAC 25. In some embodiments, the ADC 101 is implemented with the LTC1867LA 16-bit, 8-channel ADC available from Linear Technology Corporation.

As also shown in FIG. 10, some embodiments use the ADC 101 to measure the voltage on both sides of Rset. The controller 26 may then divide the voltage difference by the value of Rset to produce an estimate of the current through Rset. In some embodiments, the ADC 101 also measures the cathode voltage CATH. In some embodiments, the controller 26 uses available anode and cathode voltage measurements, together with the selected output current and Ohm's Law, to produce a rough calculation of the resistance between any two subsets of the electrodes. In some embodiments, the ADC 101 measures the battery voltage.

In some embodiments, the host 27 (see also FIG. 2) implements a graphical user interface (GUI) that provides a user with convenient access to set points, measurements and electrode configurations. An example of such a GUI is shown in FIG. 11.

Although example embodiments of the present work are described above in detail, this does not limit the scope of the present work, which can be practiced in a variety of embodiments.

What is claimed is:

1. A method of integrating transcranial direct current stimulation (tDCS) and electroencephalography (EEG), comprising:
collecting EEG data from a subject using an electrode arrangement that includes a plurality of electrodes positioned on a scalp of the subject; and
applying tDCS to the subject using said electrode arrangement.

2. The method of claim 1, including automatically alternating said applying and said collecting.

3. The method of claim 1, wherein said applying includes selecting at least two electrodes of said electrode arrangement, and automatically accessing said at least two electrodes electrically to permit tDCS current flow therebetween.

4. The method of claim 1, wherein said applying includes concurrently supplying first and second independent tDCS currents to said electrode arrangement.

5. An apparatus that integrates tDCS and EEG, comprising:
an electrode arrangement including a plurality of electrodes adapted for placement on a scalp of a subject;
an EEG analyzer coupled to said electrode arrangement to receive EEG data from the subject; and
a tDCS drive apparatus coupled to said electrode arrangement for applying tDCS to the subject.

6. The apparatus of claim 5, including a switching arrangement coupled to said electrode arrangement and said EEG analyzer and said tDCS drive apparatus, said switching arrangement configured for automatically alternating connection of electrode arrangement to said EEG analyzer and said tDCS drive apparatus.

7. The apparatus of claim 5, including a switching arrangement coupled to said electrode arrangement and said tDCS drive apparatus, said switching arrangement configured for selecting at least two electrodes from said electrode arrangement, and for automatically connecting said at least two electrodes to said tDCS drive apparatus.

8. The apparatus of claim 5, wherein said tDCS drive apparatus includes first and second current sources for respectively supplying first and second independent tDCS currents to said electrode arrangement concurrently.

9. A method of performing tDCS, comprising:
maintaining a plurality of electrodes respectively positioned at a plurality of locations on a scalp of a subject; and
during said maintaining,
using a first set of said electrodes to apply a first tDCS current flow in a first current flow configuration on the scalp, and
thereafter, using a second set of said electrodes to apply a second tDCS current flow in a second current flow configuration on the scalp, wherein said second set of electrodes is different from said first set of electrodes.

10. The method of claim 9, including, during said maintaining,
selecting said first and second sets of electrodes, and
discontinuing said using said first set of electrodes, and thereafter automatically commencing said using said second set of electrodes.

11. The method of claim 9, including collecting EEG data from the subject using the electrodes.

12. The method of claim 11, wherein said collecting includes automatically collecting said EEG data between said applications of said first and second tDCS current flows.

13. The method of claim 9, wherein, in at least one of said first and second current flow configurations, first and second independent tDCS currents are respectively supplied to first and second ones of the associated set of electrodes concurrently.

14. An apparatus for performing tDCS, comprising:
a plurality of electrodes adapted to be maintained at respective locations on a scalp of a subject;
a tDCS drive apparatus; and
a switching arrangement coupled to said electrodes and said tDCS drive apparatus for, while said electrodes are maintained at said locations,
connecting said tDCS drive apparatus to a first set of said electrodes for application of a first tDCS current flow in a first current flow configuration on the scalp, and
subsequently connecting said tDCS drive apparatus to a second set of said electrodes for application of a second tDCS current flow in a second current flow configuration on the scalp, wherein said second set of electrodes is different from said first set of electrodes.

15. The apparatus of claim 14, wherein said switching arrangement is configured for, while said electrodes are maintained at said locations,
selecting said first and second sets of electrodes, and
disconnecting said tDCS drive apparatus from said first set of electrodes, and subsequently automatically connecting said tDCS drive apparatus to said second set of electrodes.

16. The apparatus of claim 14, including an EEG analyzer coupled to said electrodes to receive EEG data from the subject.

17. The apparatus of claim 16, wherein said switching arrangement is coupled to said EEG analyzer and configured for automatically permitting said EEG analyzer to receive said EEG data between said applications of said first and second tDCS current flows.

18. The apparatus of claim 14, wherein said tDCS drive apparatus includes first and second current sources that respectively supply first and second independent currents, and wherein, in at least one of said first and second current flow configurations, said first and second independent currents are respectively applied to first and second ones of the associated set of electrodes concurrently.

19. A method of integrating tDCS and EEG, comprising:
collecting EEG data from a subject;
applying tDCS to the subject; and
automatically alternating said collecting and said applying.

20. An apparatus that integrates tDCS and EEG, comprising:
an EEG portion for receiving EEG data from the subject;
a tDCS portion for applying tDCS to the subject; and
a selecting portion for automatically alternating selection of said EEG portion and said tDCS portion for operation.

21. A method of applying tDCS to a subject, comprising:
applying a first tDCS current to the subject; and
concurrently with said applying said first tDCS current, applying to the subject a second tDCS current that is independent of said first tDCS current.

22. An apparatus for applying tDCS to a subject, comprising:
an electrode arrangement including a plurality of electrodes adapted for placement on a scalp of the subject;
a first current source connected to said electrode arrangement for supplying a first tDCS current to said electrode arrangement; and
a second current source connected to said electrode arrangement for supplying to said electrode arrangement, concurrently with said first tDCS current, a second tDCS current that is independent of said first tDCS current.

23. A headset that integrates tDCS and EEG, comprising:
a housing;
an electrode arrangement supported on said housing, said electrode arrangement including a plurality of electrodes, and said housing configured to maintain said electrodes in contact with a scalp of a subject;
an EEG analyzer supported on said housing and coupled to said electrode arrangement for collecting EEG data from the subject; and
a tDCS drive apparatus supported on said housing and coupled to said electrode arrangement for applying tDCS to the subject.

24. The headset of claim 23, including a switching arrangement coupled to said electrode arrangement and said EEG analyzer and said tDCS drive apparatus, said switching arrangement supported on said housing and configured for automatically alternating connection of said electrode arrangement to said EEG analyzer and said tDCS drive apparatus.

25. The apparatus of claim 24, wherein said switching arrangement is configured for selecting at least two electrodes from said electrode arrangement for connection to said tDCS drive apparatus.

26. The apparatus of claim 24, wherein said tDCS drive apparatus includes first and second current sources for respectively supplying first and second independent tDCS currents to said electrode arrangement concurrently.

27. The method of claim 1, wherein one of said collecting and said applying uses at least one electrode that is unused in the other of said collecting and said applying.
